(19) United States
(12) Patent Application Publication
(10) Pub. No.: US 2005/0085796 A1
(43) Pub. Date: Apr. 21, 2005

(54) Equine medicinal applicator

(76) Inventor: Jeffrey K. Wells, Conifer, CO (US)

Correspondence Address: SHERIDAN ROSS PC, 1560 BROADWAY, SUITE 1200, DENVER, CO, 80202, US

(21) Appl. No.: 10/924,483

(22) Filed: Aug. 23, 2004

Related U.S. Application Data
(60) Provisional application No. 60/497,268, filed on Aug. 22, 2003.

(51) Int. Cl. A61M 31/00; A61K 9/22

(52) U.S. Cl. 604/890.1; 604/500

(57) ABSTRACT

The present invention relates to an apparatus and method of administering an oral medication to an equine or other four legged vertebrate, and more specifically to a device adapted for use with a disposable bladder and a rotating dispensing mechanism for providing oral stimulation to the equine during administration of the medication.

### CROSS REFERENCE TO RELATED APPLICATION

This application claims priority to U.S. provisional application entitled “Equine Medicinal Applicator”, filed on Aug. 22, 2003 and having Ser. No. 60/497,268, the application being incorporated herein in its entirety by reference.

### FIELD OF THE INVENTION

The invention relates generally to an apparatus and method of administering a medicine such as a de-worming paste in commercial pre-filled syringes to coax an animal into submission for easier administration of the medication. More specifically, the invention is directed to use on equines and utilizes a rotating assembly to stimulate and promote a horse's tongue movement.

### BACKGROUND OF THE INVENTION

Horses are extremely beautiful and graceful creatures, and many people find joy and comfort breeding, training and riding horses. However, the pitfalls of owning such a creature lie in the time and expense required to keep a horse healthy. As referred to herein, the term “horses” or equine is meant to include all breeds of horses, mules, donkeys and other four-legged vertebrate used for agricultural, recreational and/or other uses, and thus may include camels, llama, cattle, bison, etc. In surveys conducted across the United States, average veterinary costs for a horse range from $150.00 to $350.00 per year. These costs reflect only the costs of preventive health care such as vaccinations, de-worming and a yearly Equine Infectious Anemia (E.I.A.) Coggins tests. The dollar value represents about 19 percent of the cost of keeping a horse for a year. If a horse contracts any disease or illness and requires medication and/or surgery, the costs could be in excess of 300 percent of the annual cost of keeping a horse. Therefore, it is important to practice preventive medicine.

Horses are capable of contracting any number of diseases, such as Botulism, Equine Encephalomyelitis, Equine Infectious Anemia, Equine Viral Arteritis (EVA), Equine Viral Rhinopneumonitis, Influenza, Potomac Horse Fever, Rabies, Strangles, or Tetanus, as well as a number of internal parasites such as tapeworms, stomach worms, pin worms, and ascarids. A horse that is not de-wormed regularly and effectively often has a dull coat, dull attitude, pot belly, persistent winter coat, more frequent episodes of colic, and is repeatedly depressed or “off” in performance. When a parasite-infested horse is fed, the resident parasite population grabs its share first. The wormy horse soon becomes debilitated. Prevention of these diseases and parasites necessitates frequent administration of vaccines, medications, and de-wormers, often through oral means. Many horse owners find that oral administration of these medicines to be an arduous task. Being forced to swallow the unpleasant-tasting medication produces irritation and fussiness in the horse, making it extremely difficult both for the owner and the horse to use the medicine. Horses will often spit the medicine back out, and with an average cost of $15-$20 per tube, multiplied by the number of times a horse must receive preventative medication, the costs of inefficient medication administration are significant, especially for owners of multiple horses. Horses are also very agile and strong animals, and it is not uncommon for an uncooperative horse to injure a caretaker while rejecting medications when being orally dispensed.

Currently, administration through oral syringes is the most common method available to owners and veterinarians. If the owner or veterinarian is not careful, or if the horse is uncooperative, most of the medicine may end up on the caretaker's sleeve, or on the ground. This method is problematic for both the horses and their owners. Much of the medicine is wasted, prompting fears of improper dosage and administration. These frequent dispensations become an arduous chore (sometimes even a fiasco) rather than simple routine. What is needed, therefore is a method of dispensing oral medications to horses such that they submit to the procedure and take up more of their medication with each administration, and so the caretaker suffers less hardship in the process.

A further problem associated with the existing medicinal applicators known in the art is the incredible amount of waste associated with the disposal of hundreds of thousands of rigid plastic medicinal containers which have a one time use, are generally not biodegradable, and thus not reusable. Furthermore, the existing medicinal applicators known in the art do not have the ability to utilize a variety of different head portions and sizes for applications in different sized animals, or animals with different oral challenges when applying an oral medication.

### SUMMARY OF THE INVENTION

In one aspect of the present invention, an apparatus is provided which orally stimulates the horse into submission and hence facilitates the horse receiving the complete dose of medication. In one embodiment, a syringe with a rolling assembly is inserted into a horse's mouth such that the horse rolls the assembly with its tongue, and lowers its head. Such stimulation is soothing for the horse, and also produces more saliva, which assists in swallowing the medicine. Further, the stimulation to the tongue promotes pushing the medicine to the rear of the mouth, and thus helps prevent the medicine being spit up and wasted. The owner can then slowly administer the medicine while the horse is in submission, making the process much more comfortable to both the horse and the caregiver. As discussed herein, “medicine” can be in the form of a liquid, paste, semi-solid, gel or combinations therein.

Thus, in one embodiment of the present invention, a therapeutic rolling assembly is provided which can be attached to the end of most conventional pre-filled syringes used in oral administration of the medication. A securement nut is used to connect the mechanism to the applicator and prevent it from coming loose inside the horse's mouth. Bearings may also be used to facilitate rotation.

In further embodiments, the rolling assembly can be flavored or textured to further stimulate the horse's oral activity. The roller itself may be edible such that the caregiver may administer the medication as the horse is chewing the mechanism. Further, the roller assembly on any other portion of the medicinal applicator preferably has a distinct smell which is favorable to the equine, and thus facilitates a position response when the medicinal applicator is presented to the equine. Further, a flavored pellet or gel may be initially discharged from the equine applicator during the initial administration of the medicine to assure a favorable taste to the equine.

Also, in another embodiment, rather than a rolling assembly that attaches to the end of most commercial syringes, the syringes themselves can come with the rollers as an integral piece, and thus do not require assembly for the mechanically challenged.

In a further embodiment, the mechanism attached to the pre-filled syringe can even be a dispensing mechanism itself such that the horse is also swallowing the medicine as it is maneuvering the mechanism with its tongue.

It is another aspect of the present invention to provide a medicinal applicator which is adapted for use with a removable bladder and thus can be used numerous times and effectively endlessly without disposal. More specifically, in an alternative embodiment of the present invention a substantially rigid container is provided which has an opening on either a forward end or rear end, and which is adapted for receiving a disposable bladder which contains the equine medicine. Upon inserting the medicinal bladder into the substantially rigid storage container, the bladder is pierced and a head portion is interconnected to the storage container and a plunger mechanism is used to provide a pressure force to the bladder, thus discharging the medicine from the bladder through the dispensing head and out a dispensing opening. The drive mechanism used to provide the pressure may utilize any types of mechanical energy devices commonly known in the art. These may include a caulking gun type of arrangement, a plunger mechanism driven by a user's finger or thumb, hydraulics, pneumatics, or any available energy source which can provide motion through a plunger assembly and thus provide pressure to the bladder within the closed container. In an alternative embodiment of the present invention the head includes a plurality of different sized interchangeable head dispensing spouts which may or may not have a roller mechanism, and may have a distinct size, flavor, shape or dispensing spout orifice depending on the application as necessary. Furthermore, the disposable bladder is preferably biodegradable and thus made out of biodegradable plastics or polymers commonly known in the art and thus may be discarded without creating waste and excessive landfill problems.

Thus, in an embodiment of the present invention, an equine medicinal applicator is provided comprising:
a storage compartment for holding medicine of said compartment having a first end and a second end;
a plunger mechanism operably interconnected to said first end of said storage compartment and capable of traveling between a first position and a second position to dispense the medicine through said dispensing spout; and
a roller means operably interconnected to at least one of said storage compartment or said dispensing spout, and which rotates around a longitudinal axis of said storage compartment, and stimulates a tongue of an equine.

### BRIEF DESCRIPTION OF THE DRAWINGS

FIG. 1 is a front elevation view of a typical medicinal paste applicator commonly used for equines;

FIG. 2 is a front elevation view of a roller apparatus and associated parts which is adapted for interconnection to the distal end of the applicator shown in FIG. 1;

FIG. 3 depicts the roller assembly of FIG. 2 with the pieces interconnected and in greater detail;

FIG. 4 depicts one embodiment of the present invention, wherein the roller assembly is selectively interconnected to the medicinal applicator shown in FIG. 1; and

FIG. 5 depicts an alternative embodiment of the present invention which is designed to receive a removable bladder while utilizing a detachable head and a plunger mechanism.

### DETAILED DESCRIPTION

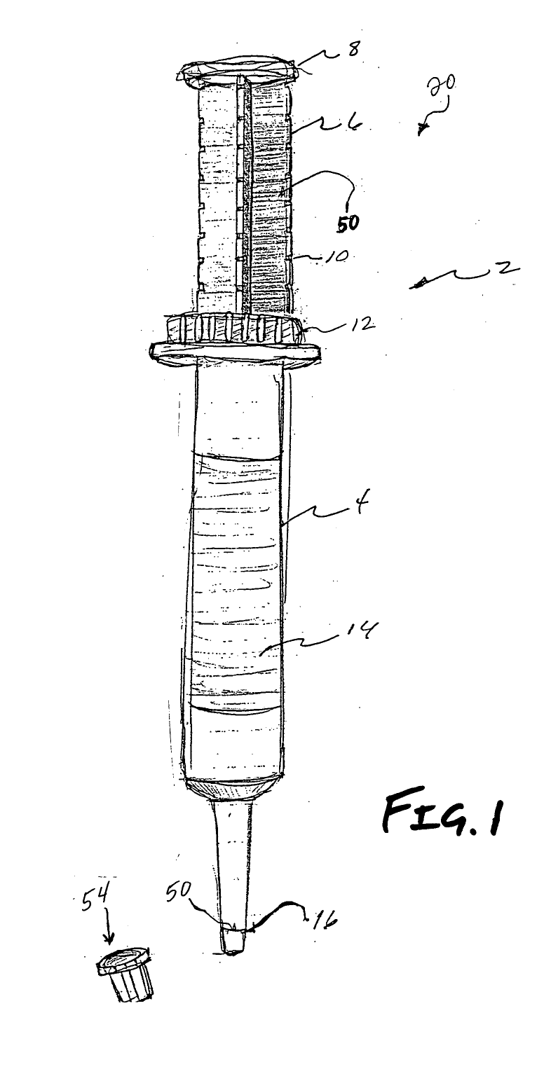

FIG. 1 is a front elevational view of the medicinal applicator 2 of the present invention which is used currently for oral administration of medication to equines. The medicinal applicator is generally comprised of a medicinal applicator body 4 which contains a specific amount of paste (depending on the type of medicine and the syringe itself), and a ribbed plunger 6 which uses air to push the paste in the applicator body 4 through the dispensing spout 16 at the end and into the mouth of the horse. A screw gauge 12 sits atop the applicator body 4 to guide the user in the proper dosage of medicine and to ensure that dosage is administered by locking the plunger 6 when the proper amount has been dispensed. These syringes make up approximately 99% of the market for oral medicinal applicators, so a roller assembly, which can be used to attach to the end of these syringes, will be extremely useful in the field. However, as will be appreciated by one skilled in the art, there are endless mechanical variations of this invention, all of which contain certain key components described below.

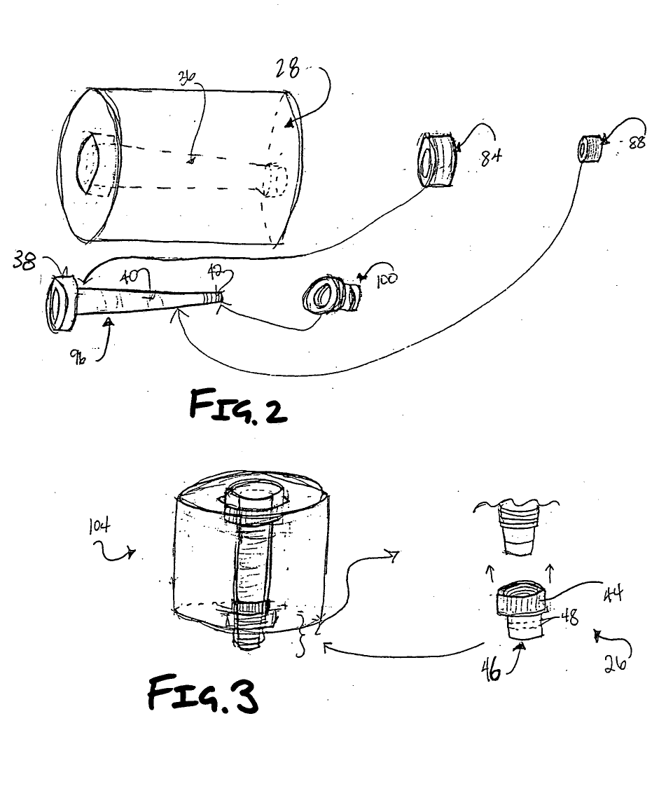

In one embodiment, the roller assembly 20 can be attached to the end of the syringe described in FIG. 1. FIG. 2 shows the internal makeup of this proposed roller assembly 20. A roller assembly cylinder 28 is constructed in a cylindrical shape. The material of this cylinder 28 can vary depending on the needs of the user and the costs associated with production. It is preferred that the material be of a soft plastic that will stimulate the horse into licking the roller assembly 20, thereby rotating it, although any number of materials may be used, including edible material. The edible material, attached to the end of the roller facilitates saliva production and swallowing in the horse such that the caregiver may slowly administer the medication as the horse is rotating the roller.

In another embodiment of the present invention, the roller can be textured to further orally stimulate the horse, or flavored to ease the medicine down the esophagus, as most commercial oral medications have unpleasant tastes to the horse. In one embodiment, a tapered cylindrical bore 36 is carved out of the roller cylinder. This bore 36 has two circular openings at each end, one being smaller than the other. The bore 36 may be in the shape of a lopsided dumbbell, as depressions must be formed at both ends to fit in the bearings used to urge rotation. These bearings are FDA-approved and may be Teflon-impregnated. Once these bearings 22 and 24 are inserted into their respective ends, a smooth tapered bore is formed to house the roller assembly interior surface 30. This interior surface 30 is crucial to the roller assembly 20 because it is used to easily fit onto the dispensing spout 16 of the medicinal applicator 2 shown in FIG. 1, ensures that rotation is smooth (when used in combination with the bearings 22 and 24), and most importantly, secures the roller assembly 20 onto the dispensing spout 16 so that it does not come loose inside the horse's mouth during operation.

FIG. 2 shows the design of the various components of one embodiment of the present invention. More specifically, the figures disclose a circular tip 38 and a smooth tapered shaft 40 with a threaded bottom end 42. The smooth shaft 40 fits perfectly inside the tapered bore created by attaching the bearings 22 and 24 to the roller cylinder 28. The threaded bottom end 42 then sticks out from the bottom end of the roller cylinder 28.

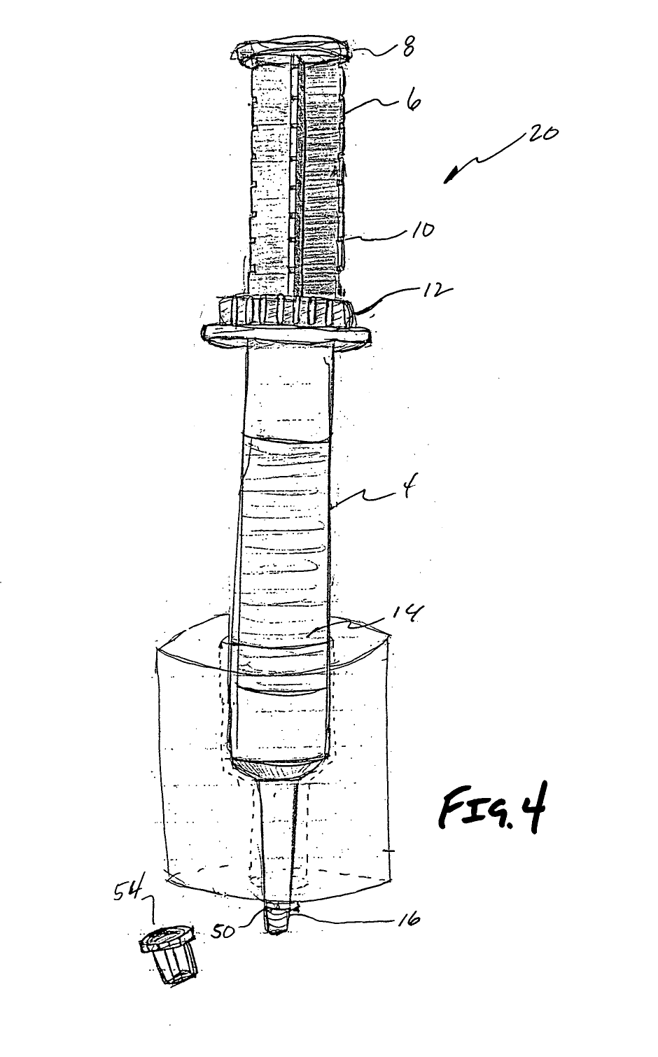

To connect the pieces of the roller assembly 20 together, a securement nut 26 may be attached to the end of the interior surface 30. The securement nut 26 is shown in FIG. 2 and FIG. 3. The top portion of the securement nut 26 has, at one end a threaded opening 44, which mates with the threaded bottom end 42 of the interior surface 30. At its bottom end 46, the securement nut 26 contains two ribbed perforations, similar to the ribbed perforations on the cap 18 of the medicinal applicator 1, which secure the roller assembly 20 to the medicinal applicator 2.

FIG. 3 shows the roller assembly 20 with the pieces interconnected. The circular tip 38 of the interior surface rests atop the top bearing 22, and by attaching the securement nut 26 at the bottom, the bearing 24 will not slip out. Thus, the roller assembly 20 is one contained unit, which can be easily disassembled and reassembled for cleaning purposes.

To attach the roller assembly 20 to the medicinal applicator 2, the user need only guide the dispensing spout 16 through the opening in the circular tip 38 of the interior surface 30 and out the other end. The ribbed perforation 50 on the end of the dispensing spout 16 should mate with the two ribbed perforations 48 on the end of the securement nut 26, and the roller assembly 20 will essentially “click” into place.

In operation, the user first inserts the apparatus 52 formed by the union of the roller assembly 20 and the medicinal applicator 2 into the mouth of the horse. The horse will begin rotation of the roller assembly 20 with its tongue and will subsequently lower its head into submission. This oral activity not only soothes the animal into submission, but also stimulates more saliva to ease the swallowing of the medicine. This production of saliva is usually followed by a swallowing action, providing an opportunity the caretaker can take to administer the medicine. Since the roller assembly 20 does not add anything extra to the bottom of the dispensing spout 16, but merely attaches to it, the user can then easily administer the medicine to the horse at that point by pushing the plunger 6 until the proper amount of medicine has been dispensed. This is the same action as performed before, only with much less commotion and mess. This method is much more preferable to the current method of administering oral medication for both the recipient and the caretaker.

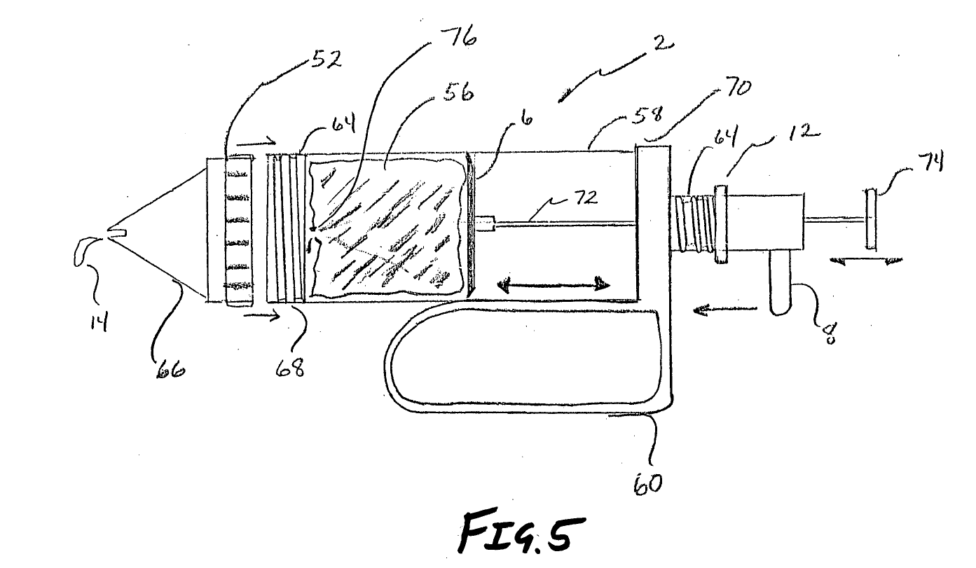

In another embodiment, the rolling assembly can simply fit upon the end of the medicinal applicator 2 in FIG. 1 like a sleeve. FIG. 4 is a front elevation view of this embodiment. The roller cylinder here is a much longer and thicker cylinder than in the previous embodiment, but bearings will no longer be used. The cylinder will extend and cover up the applicator body about ¼ of the way, as well as covering most of the dispensing spout. The inside of the roller cylinder will be shaped in the same formation as the outside of the medicinal applicator, with a slight variation in circumference at any given point of about 0.1-0.5 inches. This variation serves to provide space between the applicator and the roller so that the roller can rotate along the axis perpendicular to the applicator itself. While this system may prove more rudimentary than the previous embodiment, horses may take to it just as easily. Texture can be added to the exterior surface of the roller, which may also stimulate licking and submission in the horse. Flavors, such as oats or apples, may also be added to achieve the same effect.

To attach the rolling cylinder 28 to the applicator 2, a lid 54 is provided, similar in shape to the cap 18 that is provided with most commercial pre-filled syringes, but containing an opening at the bottom. This lid can either snap onto the end of the dispensing spout 16 in the same similar fashion that the cap 18 does, or it may be threaded on. Such a variation needs to be adapted to the medicinal applicator 2 as well. Manufacturers of the medicinal applicator 2 can create a threaded spout at the end of the dispensing spout 16 to meet with threads in the lid 54. However, it is preferred that some sort of rotational device be incorporated into the roller assembly such as the one described in the previous embodiment.

In also a different embodiment, the roller cylinder can have a plurality of small pores on its surface through which medicine can be administered slowly as the horse is licking the roller. If the cap 18 of the medicinal applicator 2 is kept on the applicator, then the action of pushing down the plunger would send the medicine through these pores and into the horse's mouth. The stimulation of saliva produced by licking the roller would ease the medication down the horse's esophagus.

There are numerous combinations of flavors, textures, lengths, widths, rotations, and designs of this roller assembly. The manufacturer may also wish to adhere the roller assembly to the pre-filled syringe before it is sold. This will save the owner the time associated with cleaning and disinfecting. One skilled in the art can appreciate the number of mechanical variations on this invention, as long one or more of the following key components are there: 1) a rotating assembly which rotates along the same axis that is perpendicular to the applicator itself, 2) a roller which is attached to the end of the medicinal applicator, 3) a roller which is manufactured with the medicinal applicator, 3) a roller which is textured in various patterns or shapes, and 4) a roller which is flavored.

Referring now to FIG. 5, an alternative embodiment of the present invention is provided herein where the medicinal applicator 2 is comprised of a substantially rigid container or storage compartment which encloses a disposable bladder which contains the equine medicine 14. More specifically, in this embodiment of the present invention a medicinal applicator 2 is designed for multiple use and for the insertion and subsequent removal of a disposable bladder 56 whenever an application of medicine is required. Preferably the disposable bladder 56 is comprised of a plastic polymer, lactide or other form of a biodegradable material which is substantially impermeable.

Thus in one embodiment the substantially rigid container 58 has a removable head 52 which is threadingly engaged to a forward end 68 of the rigid container 58. Furthermore, the interchangeable head dispensing spout 66 may be comprised of any variety of geometric shapes and may include roller assembly 20 (not shown) to provide oral stimulation to the equine. In one embodiment, the interchangeable head dispensing spout 66 is first positioned within the removable head 52 and then threadingly engaged to the container forward end 68. The rear end 70 of the medicinal applicator 2 is comprised of a handle 60 to allow the positioning of a users hand, and a thumb piece 8 which is slidingly engaged to the applicator plunger 6 by a drive rod 72. In operation, once the removable bladder 56 is positioned within the rigid container 58, the disposable bladder 56 is perforated with a knife, screwdriver or other device to allow the discharging of the medicine within the disposable bladder 56. As appreciated by one skilled in the art, the removable bladder may be inserted in the front or rear of the rigid storage compartment 58. The removable head 52 is then threadingly engaged to the rigid container front end 68, while the plunger 6 is positioned against the bladder by applying pressure to the plunger drive rod adjustment mechanism 74. The plunger drive rod 72 is operably engaged to the thumb piece 8, and may include a spring mechanism for biasing. When pressure is generally applied to the thumb piece 8 the plunger drive rod 72 pushes the plunger 6 against the removable bladder 56, thus applying pressure and discharging the medicine from the removable bladder aperture 76 into the dispensing spout 66.

As appreciated by one skilled in the art, the plunger 6 and drive mechanism used to apply pressure to the removable bladder 56 can have any variety of mechanical configurations. More specifically, a caulking gun type of ratcheting arrangement can be used wherein a user squeezes the handle to apply the necessary pressure to the plunger 6 and removable bladder 56. Preferably, the total drive distance of the plunger 6 is no more than about 3 inches which is more favorable ergonomically to a user having small hands such as women and children. Additionally, it is preferable that the handle assembly not enclose the entire hand so that it can be quickly released to prevent hand or finger injury in an equine's mouth. Furthermore, to adjust the amount of medicine for any given size of equine, a screw gauge 12 may be provided which limits the travel of the thumb mechanism 8 and thus applies only a certain amount of medicine as required. Alternatively, other types of volume control means could be used for the same purpose. Once the medicine is discharged from the removable bladder 56, the removable head 52 is threadingly disengaged from the container 58, the removable bladder 56 removed and disposed of, and the plunger 6 withdrawn towards a rear portion of the rigid container by pulling on the plunger drive rod adjustment mechanism 74. Thus, the rigid container 58 is now ready for another removable bladder 56 to be positioned within the container and the removable head 52 to be interconnected for the additional application of equine medicine.

As appreciated by one skilled in the art, it is preferable that the rigid container 58 be comprised of plastic materials which are preferably transparent to see the positioning of the bladder and the operation of the plunger, although metallic materials and other substantially rigid material including cardboard, fiberglass, and other similar materials may be used for essentially the same purpose. Preferably, the equine applicator 2 may be conveniently washed in a dishwasher, without compromising or damaging the internal components therein, and thus the equine applicator 2 can be used repeatedly without disposal of any component with the exception of the removable bladder 56. By reusing the applicator mechanism 2, it is anticipated that the cost savings will be significant since the only disposable portion is the removable bladder 56, as opposed to the current practice where the entire rigid plastic medicinal applicator is thrown away after each use.

For reference, a table is provided herein below with components and corresponding numeric labels.

| # | Component |
|---|---|
| 2 | Medicinal applicator |
| 4 | Medicinal applicator body |
| 6 | Applicator plunger |
| 8 | Thumb piece |
| 10 | Measuring indicia |
| 12 | Screw gauge |
| 14 | Medicine |
| 16 | Dispensing spout |
| 18 | Cup |
| 20 | Roller assembly |
| 22 | Tip bearing |
| 24 | End bearing |
| 26 | Securement nut |
| 28 | Roller assembly cylinder |
| 30 | Roller assembly interior surface |
| 32 | Assembly shaft |
| 36 | Tapered bore |
| 38 | Interior surface circular tip |
| 40 | Interior surface smooth tapered |
| 42 | Interior surface threaded end |
| 44 | Securement nut threaded opening |
| 46 | Securement nut bottom end |
| 48 | Ribbed perforations |
| 50 | Dispensing spout ribbed perforation |
| 52 | Removable head |
| 54 | Securement lid |
| 56 | Disposable bladder |
| 58 | Rigid storage compartment |
| 60 | Handle |
| 62 | Drive mechanism |
| 64 | Threads |
| 66 | Interchangeable head dispensing spout |
| 68 | Rigid container front end |
| 70 | Rigid container rear end |
| 72 | Plunger drive rod |
| 74 | Plunger drive rod adjustment mechanism |
| 76 | Removable bladder aperture |

The foregoing description of the present invention has been presented for purposes of illustration and description. Furthermore, the description is not intended to limit the invention to the form disclosed herein. Consequently, variations and modifications commenced here with the above teachings and the skill or knowledge of the relevant art are within the scope in the present invention. The embodiments described herein above are further extended to explain best modes known for practicing the invention and enable others skilled in the art to utilize the invention in such, or other, embodiments or various modifications required by the particular applications or uses of present invention. It is Intended that the dependent claims be construed to include all possible embodiments to the extent permitted by the prior art.

1. An equine medicinal applicator, comprising:
a storage compartment for holding medicine, said compartment having a first end and a second end;
a dispensing spout operably interconnected to said second end of said storage compartment;
a plunger mechanism operably interconnected to said first end of said storage compartment and capable of traveling between a first position and a second position to dispense the medicine through said dispensing spout; and
a roller means operably interconnected to at least one of said storage compartment or said dispensing spout, and which rotates substantially around a longitudinal axis of said storage compartment, and which orally stimulates an equine to facilitate the swallowing of the medicine when a force is applied to said plunger mechanism.

2. The medicinal applicator of claim 1, wherein said roller means is flavored.

3. The medicinal applicator of claim 1, wherein said medicinal applicator is textured.

4. The medicinal applicator of claim 1, wherein said roller means is selectively removable.

5. The medicinal applicator of claim 1, further comprising a securement means wherein said roller means is impeded from disengagement from said storage compartment.

6. The medicinal applicator of claim 1, wherein at least a part of said medicinal applicator has a smell which is pleasing to the equine.

7. The medicinal applicator of claim 1, further comprising a flavored gel or pellet which is discharged upon operation of said plunger mechanism.

8. The medicinal applicator of claim 1, further comprising a disposable pouch for retaining said medicine, said disposable pouch sized to fit within said storage compartment, wherein when pressure is applied to said disposable pouch the medicine is pushed into said dispensing spout.

9. The medicinal applicator of claim 8, further comprising a removable head interconnected to said storage compartment, wherein said disposable spout may be inserted and removed from said storage compartment.

10. The medicinal applicator of claim 8, wherein said disposable pouch is biodegradable.

11. An equine medicinal applicator, comprising:
a storage compartment for holding a medicine, said compartment having a front end and a rear end;
an opening means on at least one of said front end or said rear end of said storage compartment for receiving a medicine;
a drive means for pushing said medicine from said rear end to said front end, wherein the medicine can be discharged from a dispensing spout; and
a roller mechanism interconnected to said medicinal applicator proximate to said front end, wherein an equine can be orally stimulated while the medicine is dispensed from the dispensing spout.

12. The medicinal applicator of claim 11, wherein said drive means comprises a plunger interconnected to a handle, wherein when a force is applied to said handle the plunger moves from a rear end to a front end of said storage compartment.

13. The medicinal applicator of claim 11 wherein said medicine is stored in a disposable container which is adapted for insertion, and removal from said storage compartment.

14. The medicinal applicator of claim 13, wherein said disposable container is comprised of a flexible material which is punctured prior to dispensing to allow the medicine to be discharged from a dispensing spout located on said front end of said medicinal applicator.

15. The medicinal applicator of claim 11, further comprising a removable head assembly for selective interconnection to a front end of said storage compartment, wherein a variety of different types of roller mechanisms may be utilized with said medicinal application.

19-20. (canceled)

21. A medicinal equine applicator adapted for dispensing a medicine, comprising:
a substantially rigid storage container having at least a first opening and a front end and a rear end;
a disposable, substantially impermeable bladder adapted for retaining said disposable bladder sized for insertion into said at least said first opening of said substantially rigid storage container;
a plunger positioned within said substantially rigid storage container which is adapted to travel from said rear end to said front end and apply pressure to said disposable bladder;
a drive mechanism interconnected to said plunger, wherein when a force is applied to said drive mechanism, said plunger travels a predetermined distance; and
a removable head portion adapted for removable interconnection to said front end of said substantially rigid storage container, said removable head having an aperture for dispensing the medicine.

22. The medicinal equine applicator of claim 21, wherein said drive mechanism comprises a handle which is adapted for positioning in a user's hand and is squeezed to initiate travel in said plunger

23. The medicinal equine applicator of claim 21, wherein said drive mechanism comprises a thumb lever which is adapted for engagement.

24. The medicinal equine applicator of claim 21, wherein said plunger is interconnected to said plunger with a drive rod

25. The medicinal equine applicator of claim 21, further comprising a volume control means operably interconnected to said drive mechanism, wherein a volume of the medicine discharged from said medicinal application can be selectively adjusted.
